(19) United States
(12) Patent Application Publication
(10) Pub. No.: US 2010/0022765 A1
(43) Pub. Date: Jan. 28, 2010

(54) METHODS FOR EXTRACTING AND PURIFYING SUCRALOSE INTERMEDIATE

(76) Inventors: David Losan Ho, Los Angeles, CA (US); Zhenghao Wan, Wyuishan City (JP)

Correspondence Address: PERKINS COIE LLP, POST OFFICE BOX 1208, SEATTLE, WA, 98111-1208, US

(21) Appl. No.: 12/178,510

(22) Filed: Jul. 23, 2008

(51) Int. Cl. C07H 3/04

(52) U.S. Cl. 536/123.13

(57) ABSTRACT

The present invention provides a method for purifying sucralose-6-ester for use in making sucralose, wherein the method eliminates the need of an esterification process. In particular, ethyl acetate and ether are used to extract and purify sucralose-6-ester from a sucralose production intermediate composition comprising sucralose-6-ester.

### FIELD OF THE INVENTION

The present invention relates generally to methods for extracting and purifying sucralose intermediate, in particular, sucralose-6-ester.

### BACKGROUND OF THE INVENTION

The artificial sweetener 4,1′,6′-trichloro-4,1′,6′-trideoxygalactosucrose (“sucralose”) is derived from sucrose by replacing the hydroxyls in the 4,1′, and 6′ positions with chlorine. A number of different synthesis routes for the preparation of sucralose have been developed in which the reactive hydroxyl in the 6 position is first blocked, as by an ester group, prior to the chlorination of the hydroxyls in the 4,1′, and 6′ positions, followed by hydrolysis to remove the ester substituent to produce sucralose. Several of these synthesis routes involve tin-mediated synthesis of sucrose-6-esters.

Sucrose-6-esters may be chlorinated, such as, by the process of Walkup et al. (U.S. Pat. No. 4,980,463, which is incorporated herein by reference in its entirety). The chlorination process produces as a product a sucralose-6-ester, such as 4,1′,6′-trichloro-4,1′,6′-trideoxygalactosucrose-6-acetate, in solution in a tertiary amide, typically N,N-dimethylformamide (hereinafter, “DMF”), plus salts (produced as a result of neutralizing the chlorinating agent after completion of the chlorination reaction), chlorination reaction byproducts, and other impurities. Exemplary chlorination reaction byproducts include chlorinated carbohydrates other than sucralose, such as mono- and di-chlorinated sucrose, as well as other forms of chlorinated sucrose.

Protocols for making sucralose known in the art generally comprise an esterification process, wherein acetic anhydride and pyridine are added into sucralose-6-ester solution and the resulting product is crystallized twice in toluene to yield sucralose penta-acetate. For example, U.S. Pat. No. 4,380,476 discusses a process for the preparation of 4,1′,6′-trichloro-4,1′,6′-trideoxygalactosucrose (“TGS”), which comprises the steps of: (a) reacting sucrose with acetic anhydride in pyridine at a temperature below about −20° C. in order to obtain a mixture containing a major proportion of sucrose 6-acetate; (b) separating the sucrose 6-acetate by ion-exchange resin chromatography; (c) chlorinating the sucrose 6-acetate at the 4,1′, and 6′ positions with a reagent selected from the group consisting of a Vilsmeier reagent and sulphuryl chloride; (d) peracetylating the chlorinated product with acetic anhydride in pyridine to form TGS penta-acetate; and (d) separating and purifying the TGS penta-acetate and subsequently deacetylating the purified material to obtain TGS.

Nonetheless, sucralose production processes utilizing such esterification process are costly, both in terms of the raw materials, the equipment, and production time. And the uses of acetic anhydride, pyridine, and toluene create a variety of environmental and health concerns. Therefore, there exists a need for effective, efficient, economical, and environmentally-responsible methods for extracting and purifying sucralose-6-ester.

### SUMMARY OF THE INVENTION

Briefly described, in its preferred form, the present invention provides a method for purifying sucralose-6-ester for use in making sucralose, which comprises the steps of: extracting sucralose-6-ester from a composition comprising sucralose-6-ester using a first organic solvent (e.g., without limitation, ethyl acetate), whereby a first sucralose-6-ester solution is produced; drying/concentrating the first sucralose-6-ester solution, whereby a crude sucralose-6-ester is produced; applying water to the crude sucralose-6-ester, whereby a second sucralose-6-ester solution is produced; adding a second organic solvent (e.g., without limitation, ether) to the second sucralose-6-ester solution to precipitate sucralose-6-ester, whereby a semi-purified sucralose-6-ester is produced; heating the semi-purified sucralose-6-ester in a third organic solvent (e.g, without limitation, ethyl acetate), whereby a semi-purified sucralose-6-ester solution is produced; and cooling the semi-purified sucralose-6-ester solution, whereby a purified sucralose-6-ester is produced.

In one embodiment, the first organic solvent may be ethyl acetate. In another embodiment, the second organic solvent may be ether, such as, without limitation, diethyl ether or petroleum ether. In yet another embodiment, the first sucralose-6-ester solution may be dried/concentrated using a vacuum means. In still another embodiment, the ratio of water to the second organic solvent used in the process may be about 1:1.

Other features and advantages of the present invention will become apparent from the following detailed description. It should be understood, however, that the detailed description and the specific examples, while indicating the preferred embodiments of the present invention, are given by way of illustration only, since various changes and modifications within the spirit and scope of the present invention will become apparent to those skilled in the art from this detailed description.

### DETAILED DESCRIPTION OF THE PREFERRED EMBODIMENTS

The present invention pertains to effective, efficient, economical, and environmentally-responsible methods for extracting and purifying sucralose-6-ester for use in sucralose production, wherein the methods eliminate the need of an esterification process, which is an essential component of the sucralose production technologies currently known in the art.

In one aspect, the present invention provides a method for purifying sucralose-6-ester (e.g., without limitation, sucralose-6-acetate) for use in making sucralose, which comprises the steps of: extracting sucralose-6-ester from a composition comprising sucralose-6-ester using a first organic solvent (e.g., without limitation, ethyl acetate), whereby a first sucralose-6-ester solution is produced; drying/concentrating the first sucralose-6-ester solution, whereby a crude sucralose-6-ester is produced; applying water to the crude sucralose-6-ester, whereby a second sucralose-6-ester solution is produced; adding a second organic solvent (e.g., without limitation, ether) to the second sucralose-6-ester solution to precipitate sucralose-6-ester, whereby a semi-purified sucralose-6-ester is produced; heating the semi-purified sucralose-6-ester in a third organic solvent (e.g., without limitation, ethyl acetate), whereby a semi-purified sucralose-6-ester solution is produced; and cooling the semi-purified sucralose-6-ester solution, whereby a purified sucralose-6-ester is produced.

Sucralose-6-ester may be isolated from a composition comprising sucralose-6-ester using a first organic solvent, thereby forming a first sucralose-6-ester solution. The composition comprising sucralose-6-ester may be filtered before the organic solvent extraction to remove impurities. For example, a sucralose production intermediate mixture may be dried at about 35-75° C. (e.g., at about 35-45° C. to remove toluene and at about 65-75° C. to remove DMF) and at about −0.098 MPa. Other temperatures and pressures may also be used, such as, without limitation, a temperature of about 35° C.-45° C., about 65° C.-75° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C.; and a pressure of about −0.070 MPa to about −0.099 MPa, −0.075 MPa, −0.080 MPa, −0.085 MPa, −0.090 MPa, −0.096 MPa, or −0.098 MPa, respectively. In addition, a person skilled in the art would understand that the temperatures suitable for the purposes of the present invention may vary as a result of the changes in the pressure. For example, a higher temperature may be needed to obtain a desired result at a higher pressure, while a lower temperature may be used to obtain the same or similar result at a lower pressure. The dried intermediate mixture may be redissolved in water and the aqueous solution, which contains sucralose-6-ester, may be filtered before the organic extraction step.

Any organic solvent known in the art suitable for the purposes of the present invention may be used. In various embodiments of the present invention, the first organic solvent may be, without limitation, ethyl acetate, or a solvent having properties (e.g., polarity) similar to those of ethyl acetate, such as, without limitation, ethyl formate, butyl formate, isoamyl formate, methyl acetate, propyl acetate, butyl acetate, isobutyl acetate, amyl acetate, isoamyl acetate, hexyl acetate, benzyl acetate, methyl propionate, ethyl propionate, methyl butyrate, ethyl butyrate, butyl butyrate, amyl butyrate, isoamyl butyrate, ethyl isobutyrate, ethyl valerate, ethyl isovalerate, ethyl hexanoate, ethyl heptanoate, ethyl octanoate, ethyl nonanoate, toluene, and chloroform. In one embodiment, the first organic solvent may be ethyl acetate. In another embodiment, the composition comprising sucralose-6-ester may be extracted for multiple times and the sucralose-6-ester organic solution may be pooled to form a crude sucralose-6-ester solution for further processing.

The first sucralose-6-ester solution may be dried to produce a crude sucralose-6-ester. The terms, “dry” and “concentrate,” as used herein, refer to, without limitation, completely, substantially, or partially removing a solvent, such as, water or organic solvent (e.g., ethyl acetate) from a composition, and may be used interchangeably. Methods for drying or concentrating an organic solution are known in the art, such as, without limitation, vacuum drying, and heating. In one embodiment, the first sucralose-6-ester solution may be dried by heating in a vacuum condition. For example, the temperature of the crude sucralose-6-ester solution may be raised to about 30-75° C., about 35° C.-45° C., about 65° C.-75° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.; while pressure may be reduced to about −0.070 MPa about to about −0.099 MPa, such as, without limitation, −0.075 MPa, −0.080 MPa, −0.085 MPa, −0.090 MPa, −0.096 MPa, or −0.098 MPa.

Water may be applied to the crude sucralose-6-ester to form a second sucralose-6-ester solution. For example, a completely dried crude sucralose-6-ester may be re-dissolved in water. In another example, water may be applied to a partially dried crude sucralose-6-ester.

A second organic solvent may be applied to the second sucralose-6-ester solution to isolate sucralose-6-ester, whereby a semi-purified sucralose-6-ester is produced. Any organic solvent known in the art capable of precipitating sucralose-6-ester and suitable for the purposes of the present invention may be used. In various embodiments of the present invention, the second organic solvent may be, without limitation, one or more of an alkane, a primary ether, a secondary ether, and a tertiary ether, such as, without limitation, diethyl ether, petroleum ether, diisopropyl ether, di-tert-butyl ether, butane, isobutane, pentane, isopentane, hexane, isohexane, 3-methylpentane, heptane, 2-methylhexane, octane, 2-methylheptane, 3-methylheptane, 4-methylheptane, 3-ethylhexane, nonane, decane, and combinations thereof In one embodiment, the second organic solvent is diethyl ether and/or petroleum ether. In various embodiments of the present invention, the ratio (v/v) of the second organic solution to the second sucralose-6-ester solution may be between about 1:0.1 to about 1:2, between about 1:0.5 to about 1:1.5, about 1:0.2, about 1:0.4, about 1:0.6, about 1:0.8, about 1:1, about 1:1.2, about 1:1.4, about 1:1.6, about 1:1.8, or about 1:2.0.

The semi-purified sucralose-6-ester may be dissolved in a third organic solvent (e.g., by heating), whereby a semi-purified sucralose-6-ester solution is produced. Any organic solvent known in the art capable of dissolving sucralose-6-ester and suitable for the purposes of the present invention may be used. In various embodiments of the present invention, the third organic solvent may be, without limitation, at least one of ethyl acetate, or a solvent having properties (e.g., polarity) similar to those of ethyl acetate, such as, without limitation, ethyl formate, butyl formate, isoamyl formate, methyl acetate, propyl acetate, butyl acetate, isobutyl acetate, amyl acetate, isoamyl acetate, hexyl acetate, benzyl acetate, methyl propionate, ethyl propionate, methyl butyrate, ethyl butyrate, butyl butyrate, amyl butyrate, isoamyl butyrate, ethyl isobutyrate, ethyl valerate, ethyl isovalerate, ethyl hexanoate, ethyl heptanoate, ethyl octanoate, ethyl nonanoate, toluene, and chloroform. In one embodiment, the third organic solvent may be ethyl acetate. In various embodiments of the present invention, the ratio (v/w) of the third organic solution to the semi-purified sucralose-6-ester may be between about 1:1 to about 10:1, between about 1.5:1 to about 3:1, about 2:1, about 2.5:1, or about 5:1.

The dissolving of the semi-purified sucralose-6-ester in a third organic solvent may be facilitated by means, such as, without limitation, heating. In one embodiment, the temperature of the mixture of the semi-purified sucralose-6-ester and the third organic solvent may be raised to about 40-70° C., about 45° C.-65° C., about 50° C.-60° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., or about 65° C. The resulting semi-purified sucralose-6-ester solution may be cooled, such as, without limitation, to room temperature, thereby producing a purified sucralose-6-ester. The purified sucralose-6-ester may be filtered and dried using techniques known in the art.

The composition comprising sucralose-6-ester may be an intermediate product of sucralose production. The term, “sucralose-6-ester,” refers to any sucralose-6-ester suitable for use in the production of sucralose known in the art, such as, without limitation, sucrose-6-benzoate or sucrose-6-alkanoates (e.g., sucrose-6-acetate). For example, U.S. patent application Ser. No. 11/552,789 (hereinafter, “the '789 application”) discloses a method for making a sucralose-6-ester containing composition, the content of which is incorporated by reference herein in its entirety. According to the invention disclosed in the '789 application, a Vilsmeier reagent is prepared by adding a chlorination reagent to a solvent comprising N,N-dimethylformamide (hereinafter, “DMF”), and/or one or a mixture of other organic solvent such as toluene, cyclohexane, dichloroethane, chloroform, and carbon tetrachloride. Sucrose-6-ester is dissolved into solvent. Both the Vilsmeier reagent and the DMF solution of sucrose-6-ester are chilled to below 0° C. before mixing. The Vilsmeier reagent is then added to DMF solution of sucrose-6-ester dropwise so that the reaction temperature is kept below about 5° C. The reaction mixture is stirred at a temperature below about 5° C. for about 2 hours after the addition of Vilsmeier reagent is complete. The reaction mixture is then warmed up at room temperature and maintained at room temperature for another about 2 hours. The reaction is then heated for about 2-3 hours to reach about 110° C. and refluxed at about 110° C. for about 3 hours. Afterwards, the reaction mixture is cooled to room temperature naturally. The pH of the reaction mixture is first adjusted to 8-9, and then to 6-7. After removing most of the solvent by distillation under reduced pressure, the sucrose-6-ester is extracted by ethyl acetate and water. The combined organic phase is distilled under reduced pressure to afford sucralose-6-ester syrup.

In various embodiments, a chlorination reagent can also be dissolved in one or a plurality of organic solvents, such as toluene, cyclohexane, dichloroethane, chloroform, and carbon tetrachloride, before it is added into the DMF solution of sucrose-6-acetate with the same protocol as described above. The chlorination reagent may be selected from a group consisting of triphenylhydrazine, phosphoric chloride, thionyl chloride, phosgene, oxalyl chloride. In one embodiment, the chlorination reagent may be triphosgene (Bis(trichloromethyl) carbonate, BTC). BTC is generally considered to be safe and convenient to use, and it causes no or little pollution and corrosion concerns.

In one embodiment, the concentration of the sucrose-6-ester may be at about 0.1 mol/L. In another embodiment, the mole equivalent of chlorination reagent comparing to sucrose-6-ester may range from about 2.8 to about 3.5.

The reaction may be carried out under vacuum to avoid the oxidation of the reaction mixture by oxygen in ambient atmosphere. Alternatively undesired oxidation may be avoided by refluxing the reaction mixture in the presence of a low-boiling-point organic solvent such as cyclohexane, dichloroethane, ethyl acetate, chloroform and carbon tetrachloride.

Sucrose-6-ester may be produced using methods known in the art which are suitable for the purposes of the present invention, such as, without limitation, the method disclosed in U.S. patent application Ser. No. 11/552,813 (hereinafter, “the '813 application”), the content of which is incorporated by reference herein in its entirety. According to the invention disclosed in the '813 application, a process for the synthesis of sucrose-6-ester from sucrose comprises reacting a mixture comprising sucrose, an ester, and an organic solvent with a solid super acid catalyst for a period of time and at a temperature sufficient to produce sucrose-6-ester. The catalyst is then filtered and can be reused for the same reaction. The ester is distilled to afford a mixture comprising sucrose-6-ester and the organic solvent. If the organic solvent is one that is compatible for the chlorination reaction, the obtained sucrose-6-ester solution can be used for the next step in sucralose synthesis without further purification.

The choice of organic solvents is determined by the solubility of the sucrose and the ester in the solvents, as well as the safety and toxicity considerations. The solvent may be a polar organic solvent, such as, without limitation, DMF. The amount of the organic solvent to be used may be determined using techniques known in the art in view of the solvent used and purposes of the invention. When the polar solvent is DMF, it may be used in an amount of approximately 5 ml/g sucrose.

The amount of the ester to be used will be determined to facilitate the conversion of the desired sucrose-6-ester and suppress the formation of outgrowth. When the ester is EtOAc, it may be used in an amount of from about 5 to about 12 mol/mol sucrose.

The solid super acid catalyst may be selected from a group containing one or a mixture of sulfated oxide of an element selected from those of group 3, group 4, group 5, group 6, group 7, group 8 group 9, group 10, group 11, group 12, group 13, group 14, group 15 of the periodic table and those of the series of lanthanides, alone or combined with each other. Examples of solid super acid catalyst include, without limitation, SO₄²—TiO₂/Al₂O₃, SO₄²—Fe₂O₃/Al₂O₃, SO₄²—ZnO/Al₂O₃, SO₄²—CeO₂/Al₂O₃, SO₄²—ZrO₂/Al₂O₃, SO₄²—TiO₂/Al₂O₃ or SO₄²—TiO₂. In one embodiment of the invention, a one-step synthesis of sucrose-6-acetate comprises selective esterification with EtOAc at the 6-position of sucrose in the presence of solid super acid such as SO₄²—TiO₂/Al₂O₃ or SO₄²—TiO₂.

The present invention is described in the following Examples, which are set forth to aid in the understanding of the invention, and should not be construed to limit in any way the scope of the invention as defined in the claims which follow thereafter.

### EXAMPLES

34 g of sucrose was used to make sucrose-6-acetate, which was then reacted with triphosgene. The pH of the reaction mixture was first adjusted to 8-9, and then to 6-7. The reaction mixture was heated to 65° C. using a water bath and dried at −0.098 MPa. The dried sucralose intermediates were re-dissolved in 200 ml water, and filtered. The aqueous solution was extracted by using ethyl acetate (200 ml, 6×) and the ethyl acetate fractions were pooled.

The pooled ethyl acetate fractions were dried at −0.098 MPa and 35° C. The dried substance was re-dissolved in 50 ml water. 50 ml of ether was added to the aqueous solution to precipitate sucralose-6-acetate, which was filtered and collected. Yield: 15-18 gram semi-purified sucralose-6-acetate.

The semi-purified sucralose-6-acetate was re-dissolved in 2.5 times (volume to weight) ethyl acetate at 60° C. The sucralose-6-acetate ethyl acetate solution was slowly cooled. After 6 hours, the sucralose-6-acetate was filtered and vacuum dried at 50° C. Yield: 8-10 gram sucralose-6-acetate crystal, which contained 98% of sucralose-6-acetate.

While the invention has been disclosed in its preferred forms, it will be apparent to those skilled in the art that many modifications, additions, and deletions can be made therein without departing from the spirit and scope of the invention and its equivalents as set forth in the following claims.

What is claimed is:

1. A method for purifying sucralose-6-ester for use in making sucralose, comprising:
extracting sucralose-6-ester from a composition comprising sucralose-6-ester using a first organic solvent, whereby a first sucralose-6-ester solution is produced;
drying the first sucralose-6-ester solution, whereby a crude sucralose-6-ester is produced;
applying water to the crude sucralose-6-ester, whereby a second sucralose-6-ester solution is produced;
adding a second organic solvent to the second sucralose-6-ester solution to precipitate sucralose-6-ester, whereby a semi-purified sucralose-6-ester is produced;
heating the semi-purified sucralose-6-ester in a third organic solution, whereby a semi-purified sucralose-6-ester solution is produced; and
cooling the semi-purified sucralose-6-ester solution, whereby a purified sucralose-6-estate is produced.

2. The method of claim 1, wherein the first organic solvent is ethyl acetate.

3. The method of claim 1, wherein the second organic solvent is an ether or alkane.

4. The method of claim 1, wherein the second organic solvent is ethyl ether, petroleum ether, or a combination thereof.

5. The method of claim 1, wherein the third organic solvent is ethyl acetate.

6. The method of claim 1, wherein the first sucralose-6-ester solution is dried using vacuum drying.

7. The method of claim 1, wherein the ratio (v/v) of the second organic solvent to the second sucralose-6-ester solution is about 1:0.1-2.

5. The method of claim 1, wherein the semi-purified sucralose-6-ester is heated at about 45-65° C. in ethyl acetate.

6. The method of claim 1, wherein the sucralose-6-ester is sucrose-6-benzoate or sucralose-6-acetate.
